(19) United States
(12) Patent Application Publication
(10) Pub. No.: US 2022/0340647 A1
(43) Pub. Date: Oct. 27, 2022

(54) MEDICINAL PREPARATION FOR TREATING FIBROSIS WITH ANTI BSP ANTIBODIES

(71) Applicant: IMMUNDIAGNOSTIK AG, Bensheim (DE)

(72) Inventors: Franz Paul ARMBRUSTER, Bobenheim-Roxheim (DE); Christoph REICHETZEDER, Potsdam (DE)

(73) Assignee: IMMUNDIAGNOSTIK AG, Bensheim (DE)

(21) Appl. No.: 17/642,507

(22) PCT Filed: Sep. 10, 2020
(86) PCT No.: PCT/EP2020/075339

§ 371 Date: Mar. 11, 2022

(30) Foreign Application Priority Data
Sep. 14, 2019 (DE) 10 2019 124 768.3

(51) Int. Cl. C07K 16/18; A61P 9/00; A61P 13/12

(52) U.S. Cl. CPC C07K 16/18; A61P 9/00; A61P 13/12; A61K 2039/505

(57) ABSTRACT

A composition comprising a monoclonal antibody against bone sialoprotein (BSP) for use in a therapy of cardioprotection in a subject suspected of suffering from fibrosis, whereby a pathological accumulation of collagen and/or the progression of fibrosis are prevented.

### FIELD OF THE INVENTION

The present invention relates to medicinal preparations containing antibodies for use in therapy and, in particular, for treating fibrosis.

### BACKGROUND OF THE INVENTION

Upon injury or insult, the organism activates a process of tissue repair which involves a fine regulation of extracellular matrix (ECM) synthesis and degradation, thereby ensuring maintenance of normal tissue architecture. However, if the tissue injury is severe or repetitive, or if the wound healing response itself becomes deregulated, a progressive irreversible fibrotic response can occur. Fibrosis is the formation of excess fibrous connective tissue in an organ or tissue in a reparative or reactive process, which may interfere with or even alter the normal architecture and function of the underlying organ or tissue. Fibrosis can be defined as the pathological state of excess deposition of fibrous tissue and it involves the pathological accumulation of extracellular matrix components, such as collagen. This may result in scarring and thickening of the affected tissue which interferes with the normal functioning of the organ.

Collagen is the main structural protein component of connective tissue. It is abundant in the extracellular space within the various connective tissues and, in mammals it contributes to 25% to 35% of the whole-body protein content. Collagen consists of amino acids bound together to form triple-helices of elongated fibrils, conferring great tensile strength. In fact, this protein is the main component of fascia, cartilage, ligaments, tendons, bone and skin.

Cardiac fibrosis is a hallmark of heart disease and it is thought to contribute to sudden cardiac death, ventricular tachyarrhythmia, left ventricular (LV) dysfunction, and heart failure. Cardiac fibrosis is characterized by a disproportionate accumulation of fibrillated collagen that may occur after myocyte death, inflammation, enhanced workload, hypertrophy, and stimulation by a number of hormones, cytokines, and growth factors. Cardiac fibrosis may also refer to an abnormal thickening of the heart valves due to inappropriate proliferation of cardiac fibroblasts but more commonly the term refers to the proliferation of fibroblasts in the cardiac muscle. Fibroblasts normally secrete collagen, and function to provide structural support for the heart. When over-activated, this process causes thickening and fibrosis of the valve, with white tissue building up primarily on the tricuspid valve, but also occurring on the pulmonary valve. The thickening and loss of flexibility eventually may lead to valvular dysfunction and right-sided heart failure.

Stopping the stimulation or production of serotonin has been proposed as a treatment for cardiac valve fibrosis or fibrosis in other locations. Surgical tricuspid valve replacement for severe stenosis (blockage of blood flow) has been necessary in some patients. Also, a compound found in red wine, resveratrol, has been suggested to slow the development of cardiac fibrosis (Olson et al. (2005) “Inhibition of cardiac fibroblast proliferation and myofibroblast differentiation by resveratrol”. American journal of physiology. Heart and circulatory physiology 288 (3): H1131-8; Aubin, et al. (2008) “Female rats fed a high-fat diet were associated with vascular dysfunction and cardiac fibrosis in the absence of overt obesity and hyperlipidemia: Therapeutic potential of resveratrol”. The Journal of Pharmacology and Experimental Therapeutics 325 (3): 961-8.) Attempts for countering cardiac fibrosis relying on microRNA inhibition (miR-21, for example) showed no conclusive data. A number of different approaches addressing the prevention or treatment of cardiac fibrosis are disclosed, for example, in EP18730384, EP17759189, EP16864627 and EP16169795.

However, no medication is available on the market to effectively prevent or treat cardiac fibrosis, so that there is a need to develop effective pharmaceutical preparations to treat this disorder. The state of the art therefore represents a problem.

### SUMMARY OF THE INVENTION

The present disclosure relates to a medicinal preparation comprising a monoclonal antibody against bone sialoprotein (BSP) for use in a therapy of cardioprotection in a subject diagnosed or suspected of suffering from fibrosis, whereby a pathological accumulation of collagen and/or the progression of fibrosis are prevented.

In a preferred embodiment, the subject may be human and suffer from early/mid stage chronic kidney disease.

In one aspect, the subject may be suspected of suffering from cardiac fibrosis.

In another aspect of the disclosure, the therapy of cardioprotection may be directed to prevent fibrosis and/or accumulation of collagen in the myocardium.

In one aspect of the disclosure, the subject may suffer from uremic calcification.

In another aspect, the subject does not suffer from hypertension.

In one embodiment, the medicinal preparation may be used in a therapeutically effective amount between 0.1 to 10 mg/kg body weight, preferably 1 to 5 mg/kg body weight, more preferably 2.5 to 3.5 mg/kg body weight.

In another embodiment, the medicinal preparation may comprise a pharmaceutically acceptable vehicle and be administered subcutaneously.

In one aspect of the disclosure, the antibody may be a rat monoclonal antibody or a humanized monoclonal antibody.

In another aspect, a kit is disclosed comprising a monoclonal antibody against bone sialoprotein (BSP) for use in a therapy of cardioprotection in a subject diagnosed or suspected of suffering from fibrosis, wherein the monoclonal antibody is comprised in a medicinal preparation as described above.

Further embodiments and advantages of the invention will become apparent from the examples and drawings, which shall illustrate and explain the invention. The desire scope of protection has been defined in the claims.

### BRIEF DESCRIPTION OF THE DRAWINGS

In the figures and drawings appended hereto:

FIG. 1 shows a schematic representation of the study protocol analyzing healthy vehicle-treated control animals (CNT); adenine-induced CKD vehicle-treated animals; and adenine-induced CKD animals treated with anti-BSP-antibody in low (CKD+LD Anti-BSP), medium (CKD+LD Anti-BSP), and high concentrations (CKD+HD Anti-BSP).

FIG. 2 shows a graphic representation of the food intake (FIG. 2A) and body weight (FIG. 2B) changes throughout the study analyzing vehicle-treated control animals (CNT); adenine-induced CKD vehicle-treated animals; and adenine-induced CKD animals treated with anti-BSP-antibody in low (CKD+LD Anti-BSP), medium (CKD+LD Anti-BSP), and high concentrations (CKD+HD Anti-BSP).

FIG. 3 is a bar diagram representing the quantification of cardiac interstitial collagen content investigated by analyzing microscopic images of Sirius red stained heart sections from healthy vehicle-treated control animals (CNT), adenine-induced CKD vehicle-treated animals (CKD), and adenine-induced CKD animals treated with Anti-BSP-antibody in low (CKD+LD Anti-BSP), medium (CKD+MD Anti-BSP), and high concentrations (CKD+HD Anti-BSP).

FIG. 4 shows representative images of Sirius red-stained myocardium sections from healthy vehicle-treated control animals (CNT), adenine-induced CKD vehicle-treated animals (CKD), and adenine-induced CKD animals treated with Anti-BSP-antibody in low (CKD+LD Anti-BSP), medium (CKD+MD Anti-BSP), and high concentrations (CKD+HD Anti-BSP).

### DETAILED DESCRIPTION OF THE INVENTION

The disclosure describes compositions and a kit for use in a therapy of cardioprotection in a subject suspected of suffering from fibrosis, whereby the pathological accumulation of collagen and/or the progression of fibrosis are prevented. In one embodiment, the composition for use in a therapeutically effective amount is preferably between 0.1 to 10 mg/kg body weight, preferably 1 to 5 mg/kg body weight, more preferably 2.5 to 3.5 mg/kg body weight. The compound is preferably for subcutaneous use. The subject is preferably a human suffering from early/mid stage chronic kidney disease.

Many animal models have been developed to study the causes and treatments of chronic kidney disease (CKD) in humans, an insidious disease resulting from kidney injury and characterized by persistent functional decline for more than 3 months, with or without evidence of structural deficit. The eventual outcome of CKD may be end-stage kidney disease (ESKD), where patients need dialysis or transplantation to survive. Cardiovascular disease is accelerated in patients with CKD and contributes to increased mortality, with the relationship between CKD and cardiovascular disease being bi-directional. Most animal models do not mimic the complexity of the human disease. For example, the 5/6 nephrectomy model relies on the unilateral nephrectomy and either partial infarction or amputation of the poles of the remaining kidney. The pathological features in the kidney include tubulointerstitial atrophy, focal hypertrophy, and glomerulosclerosis, with cardiovascular changes such as hypertension, cardiac hypertrophy, inflammation and fibrosis.

The adenine diet model of CKD in rodents is an exception. The original adenine diet model produced rapid-onset kidney disease with extensive tubulointerstitial fibrosis, tubular atrophy, crystal formation and marked vessel calcification. Since then, lower adenine intake in rats has been found to induce slowly progressive kidney damage and cardiac disorders. These chronic adenine diet models allow the characterization of relatively stable kidney and cardiovascular disease, similar to CKD in humans. In addition, interventions for reversal can be tested. In summary, the data presented here support the use of chronic low-dose adenine diet in rats as an easy and effective model for developing a therapeutic strategy directed to an early/mid stage of human CKD, with special focus on the myocardial fibrosis. This model can thus be used to test therapies that may reverse or prevent progression of cardiac fibrosis, in particular, myocardial fibrosis. We developed a model that more closely mimicked the slow progression of human CKD as well as showing pathological changes in the myocardial system to demonstrate the close relationship between cardiac fibrosis and CKD in humans.

In our model, systolic blood pressure was normal and remained stable for the duration of the study, thereby allowing the monitoring of the fibrotic process in the myocardium, without the intervention of hypertension and cardiovascular events in the pathological process. This model mimics therefore the situation of a patient suffering from CKD, however, at a stage in which an increased blood pressure or hypertension is not yet taking place.

Chronic kidney disease (CKD) refers to all 5 stages of kidney damage, from very mild damage in Stage 1 to complete kidney failure in Stage 5. The stages of chronic kidney disease are based on how well the kidneys can perform their function, namely filtration of waste products and extra fluids from the blood. In the early stages of kidney disease, the kidneys are still able to filter out waste from the blood. In the later stages of CKD, the kidneys are so deteriorated that they may stop functioning altogether.

At Stages 1 and 2 CKD there is mild kidney damage, and usually no obvious symptoms. Usually, an eGFR (estimated glomerular filtration rate) between 60 and 89 means the kidneys are healthy and functioning normally. At Stage 2 CKD, despite having a normal eGFR, other signs of kidney damage, such as the presence of protein in urine or physical damage to the kidneys and other organs may however occur.

At Stage 3 CKD, an eGFR between 30 and 59 means that the kidneys are moderately damaged and not working properly. Stage 3 CKD is separated into two stages; Stage 3a and Stage 3b. Stage 3a is characterized by an eGFR between 45 and 59, and Stage 3b by an eGFR between 30 and 44. By Stage 3 CKD, patients are likely to have health complications as a result of waste building up in the organism. Common complications from kidney disease at this stage are high blood pressure (hypertension), anemia, and bone disease. Of note, when the kidneys are malfunctioning, the hormone system regulating blood pressure becomes misbalanced, which in turn causes an increased demand of heart pumping performance (pressure overload) in order to increase blood supply to the kidneys. A prolonged period of misbalance will inevitably lead to heart disease.

The present disclosure is directed to the provision of an animal model which mimics the stage between CKD stage 2, where organs may be already damaged and/or fibrotic, however, without suffering from hypertension, and CKD Stage 3, when hypertension contributes to the general pathology. Following this approach, the fibrotic process can be treated in isolation without the intervention of cardiovascular processes which are not to be treated as such by use of the present composition.

At Stage 4 CKD, an eGFR between 15 and 30 indicates that the kidneys are moderately or severely damaged and poorly functioning. Stage 4 CKD is the last stage of kidney disease before kidney failure. At Stage 4 CKD, concomitant high blood pressure, anemia, and bone disease worsen. Stage 5 CKD is characterized by an eGFR less than 15. At Stage 5 CKD, the kidneys are close to failure or have completely failed.

The term “cardiac fibrosis” or “heart fibrosis” commonly refers to the proliferation of fibroblasts and the pathological deposition or accumulation of extracellular matrix proteins, such as collagen, in the cardiac muscle. This term may also describe an abnormal thickening of the heart valves due to inappropriate proliferation of cardiac fibroblasts. Fibroblasts normally secrete collagen, and function to provide structural support for the heart. When over-activated, this process causes thickening and fibrosis of the myocardium and valves with white tissue building up. The thickening and loss of flexibility eventually may lead to valvular dysfunction and right-sided heart failure. Chronic kidney disease is associated with fibrosis in different tissues. Without wishing to be bound by theory, the present disclosure describes an animal model of chronic kidney disease corresponding with an early or mid stage CKD in humans, characterized by renal malfunctioning however lacking hypertension or associated cardiovascular symptoms. The present animal model, in contrast to 5/6-Nx or sham operated rats, is characterized by the occurrence of fibrosis at an early/mid CKD stage, notably, without a hypertension phenotype and the mechanical damage of the heart due to increased pumping performance. This allows the design of a therapy directed to the prevention of the pathological collagen deposition and/or fibroblast activity leading to heart tissue damage, in particular, the myocardium.

The disclosure describes compositions for antagonizing collagen secretion or collagen deposition in the heart, in particular, the myocardium of a human subject comprising the administration of a therapeutically effective amount of the composition of the disclosure to the human subject in need thereof. According to the present disclosure, the excessive collagen secretion or deposition in the myocardium occurs in association with an early/mid CKD stage phenotype without suffering from hypertension.

The compositions of the disclosure have beneficial pharmaceutical properties and may be applied as pharmaceutical applications for use in the prevention of cardiac fibrosis and/or a therapy of cardioprotection in subjects suffering from early/mid CKD stage without hypertension.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are well known by physicians.

The terms “treatment” or “therapy” of a subject includes the application or administration of a composition described herein to a subject for use in delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition.

Cardioprotection includes all mechanisms and means that contribute to the preservation of the heart's function and structural integrity by reducing or even preventing myocardial damage. According to this definition, cardioprotection in a subject can be addressed by therapeutic approaches comprising the use of the composition of the disclosure. The major aim of an acute or chronic cardioprotective intervention or therapy is to prevent the loss of functional myocardium and, thus, preserve ventricular function.

Bone sialoprotein (BSP) is a component of mineralized tissues such as bone, dentin, cementum and calcified cartilage. BSP is a significant component of the bone extracellular matrix and has been suggested to constitute approximately 8% of all non-collagenous proteins found in bone and cementum. BSP, a SIBLING protein, was originally isolated from bovine cortical bone as a 23-kDa glycopeptide with high sialic acid content. The human variant of BSP is called bone sialoprotein 2 also known as cell-binding sialoprotein or integrin-binding sialoprotein and is encoded by the IBSP gene.

As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g., bioavailability, stability, potency, toxicity, etc.), and the particular disorder(s) the subject is suffering from. The therapeutically effective amount will also vary according to the severity of the disease state, organ function, or underlying disease or complications.

“Pharmaceutically acceptable vehicle” refers to a diluent, adjuvant, excipient, or carrier with which a compound is administered. The term “pharmaceutically acceptable” refers to drugs, medicaments, inert ingredients etc., which are suitable for use in contact with the tissues of humans and other animals without undue toxicity, incompatibility, instability, irritation and allergic response, commensurate with a reasonable benefit/risk ratio.

Cardiac fibrosis is characterized by net accumulation of extracellular matrix proteins in the cardiac interstitium and contributes to both systolic and diastolic dysfunction in many cardiac pathophysiologic conditions. Although activated myofibroblasts are the main effector cells in the fibrotic heart, monocytes/macrophages, lymphocytes, mast cells, vascular cells and cardiomyocytes may also contribute to the fibrotic response by secreting key fibrogenic mediators. Inflammatory cytokines and chemokines, reactive oxygen species, mast cell-derived proteases, endothelin-1, the renin/angiotensin/aldosterone system, matricellular proteins and growth factors (such as TGF-β and PDGF) are some of the best studied mediators implicated in cardiac fibrosis. Both experimental and clinical evidence suggests that cardiac fibrotic alterations may be reversible at an early stage of the disease. However, the mechanisms responsible for initiation, progression and resolution of cardiac fibrosis are not fully understood. It is therefore crucial to design anti-fibrotic treatment strategies for patients with heart disease, in particular, suffering from CKD at early/mid stages without hypertension, before heart damage is irreversible.

Because the adult mammalian myocardium has negligible regenerative capacity, the most extensive fibrotic remodeling of the ventricle is found in diseases associated with acute cardiomyocyte death. For example, following acute myocardial infarction, sudden loss of a large number of cardiomyocytes triggers an inflammatory reaction, ultimately leading to replacement of dead myocardium with a collagen-based scar. Several other pathophysiologic conditions induce more insidious interstitial and perivascular deposition of collagen, in the absence of completed infarction. Aging is associated with progressive fibrosis that may contribute to the development of diastolic heart failure in elderly patients. Pressure overload, induced by hypertension or aortic stenosis, results in extensive cardiac fibrosis that is initially associated with increased stiffness and diastolic dysfunction; a persistent pressure load may eventually lead to ventricular dilation and combined diastolic and systolic heart failure.

In the adult mammalian heart, ventricular myocytes are arranged in layers of tightly coupled cardiomyocytes; adjacent layers are separated by clefts. The laminar architecture of the myocardium is defined by an intricate network of extracellular matrix proteins, comprised primarily of fibrillar collagen. Based on morphological characteristics, the cardiac matrix network can be subdivided into three constituents: the epi-, peri- and endomysium. The epimysium is located on the endocardial and epicardial surfaces providing support for endothelial and mesothelial cells. The perimysium surrounds muscle fibers, and perimysial strands connect groups of muscle fibers together. The endomysium arises from the perimysium and surrounds individual muscle fibers. Endomysial struts tether muscle fibers together and to their nutrient microvasculature and function as the sites for connections to cardiomyocyte cytoskeletal proteins across the plasma membrane. The collagen-based cardiac matrix network does not only serve as a scaffold for the cellular components, but is also important for transmission of the contractile force. Approximately 85% of total myocardial collagen is type I, primarily associated with thick fibers that confer tensile strength. Type III collagen, on the other hand, represents 11% of the total collagen protein in the heart, typically forms thin fibers, and maintains the elasticity of the matrix network. In addition to collagens, the cardiac extracellular matrix also contains glycosaminoglycans (such as hyaluronan), glycoproteins and proteoglycans. Significant stores of latent growth factors and proteases are also present in the cardiac extracellular matrix; their activation following injury may trigger the fibrotic response.

The cardiac interstitium contains several distinct cell types. Cardiac fibroblasts are enmeshed in the endomysial interstitial matrix that surrounds cardiomyocytes and represent the most abundant interstitial cells in the adult mammalian heart. In the developing heart, cardiac fibroblasts regulate cardiomyocyte proliferation through a fibronectin/β1-integrin mediated pathway. As the predominant matrix-producing cells in the myocardium, fibroblasts play an important role in preserving the integrity of the matrix network. The cardiac fibroblast population undergoes a dramatic change during the neonatal period. As the fetal circulation transitions to the neonatal circulation, elevated left ventricular pressures trigger a marked expansion of the cardiac fibroblast population within the first two neonatal weeks. In the young adult heart, cardiac fibroblasts remain quiescent and do not exhibit significant inflammatory or proliferative activity. Vascular cells (smooth muscle cells, endothelial cells and pericytes) are also present in the cardiac interstitium; relatively small numbers of mast cells and macrophages also reside in the mammalian heart, usually localized around vessels.

Mature fibrillar collagen is highly stable with a half-life of 80-120 days. Collagen turnover in the normal heart is primarily regulated by resident cardiac fibroblasts. Homeostatic control of the cardiac extracellular matrix involves ongoing synthesis and degradation of matrix proteins. Disturbance of the tightly regulated balance between the synthetic and degradative aspects of collagen metabolism results in profound structural and functional abnormalities of the heart. Fibrosis disrupts the coordination of myocardial excitation/contraction coupling in both systole and diastole and may result in profound impairment of systolic and diastolic function. Increased deposition of interstitial collagen in the perimysial space is initially associated with a stiffer ventricle and diastolic dysfunction. However, active fibrotic remodeling of the cardiac interstitium is also associated with matrix degradation leading to the development of ventricular dilation and systolic failure. Disturbance of the collagen network in the fibrotic heart may cause systolic dysfunction through several distinct mechanisms. First, loss of fibrillar collagen may impair transduction of cardiomyocyte contraction into myocardial force development resulting in uncoordinated contraction of cardiomyocyte bundles. Second, interactions between endomysial components (such as laminin and collagen) and their receptors may play an important role in cardiomyocyte homeostasis. Laminin α4 chain deficient mice exhibit microvascular abnormalities leading to systolic ventricular dysfunction, suggesting a link between defects in the matrix network and the structural integrity of the myocardium. Finally, fibrosis may result in sliding displacement (slippage) of cardiomyocytes leading to a decrease in the number of muscular layers in the ventricular wall and subsequent left ventricular dilation. Beyond its profound effects on cardiac function, fibrotic ventricular remodeling also promotes arrhythmogenesis through impaired conduction and subsequent generation of reentry circuits.

Regardless of the pathophysiologic mechanisms responsible for development of the fibrotic response, cardiomyocyte death is often the initial event responsible for activation of fibrogenic signals in the myocardium. In other cases, injurious stimuli (such as pressure overload or myocardial inflammation) may activate profibrotic pathways in the absence of cell death. Several cell types are implicated in fibrotic remodeling of the heart either directly by producing matrix proteins (fibroblasts), or indirectly by secreting fibrogenic mediators (macrophages, mast cells, lymphocytes, cardiomyocytes and vascular cells). The relative contribution of the various cell types is often dependent on the underlying cause of fibrosis. However, in all conditions associated with cardiac fibrosis, fibroblast transdifferentiation into secretory and contractile cells, termed myofibroblasts, is the key cellular event that drives the fibrotic response.

Myofibroblasts are phenotypically modulated fibroblasts that accumulate in sites of injury and combine ultrastructural and phenotypic characteristics of smooth muscle cells, acquired through formation of contractile stress fibers, with an extensive endoplasmic reticulum, a feature of synthetically active fibroblasts. Expression of α-smooth muscle actin (α-SMA) identifies differentiated myofibroblasts in injured tissues, but is not a requirement for the myofibroblast phenotype.

Regardless of the etiology of cardiac injury, myofibroblasts are prominently involved in both reparative and fibrotic processes. Increased myofibroblast accumulation in the cardiac interstitium has been reported, not only in myocardial infarction, but also in the pressure and volume overloaded myocardium, in the aging heart and in alcoholic cardiomyopathy. The origin of myofibroblasts in the fibrotic heart remains controversial. The abundance of fibroblasts in the normal myocardium and the marked induction of mediators that promote myofibroblast transdifferentiation following cardiac injury (such as TGF-β1 and ED-A fibronectin) suggest that activation of resident cardiac fibroblasts may represent the most important source of myofibroblasts in the fibrotic heart. Moreover, proliferating myofibroblasts are commonly found in large numbers in infarcted hearts. Studies in human patients with cardiac fibrosis due to chronic transplant rejection demonstrated that most of the collagen deposited in fibrotic human hearts is derived from cells of intracardiac origin.

Increased accumulation of fibrillar collagen in the cardiac interstitium is the hallmark of cardiac fibrosis. Synthesis of both type I and type III collagen is markedly increased in the remodeling fibrotic heart regardless of the etiology of fibrosis. In models of hypertensive cardiac fibrosis and of myocardial infarction, type I collagen exhibits more intense and prolonged upregulation than collagen III. However, in patients with ischemic cardiomyopathy the ratio of collagen I:collagen III synthesis was decreased suggesting that expression patterns of various collagen isoforms in the fibrotic heart may depend on contextual factors. Activated myofibroblasts are the main cellular sources of collagens in the fibrotic heart; once outside the cell procollagen chains are processed, assembled into fibrils and cross-linked. Collagen cross-linking is associated with the development of diastolic dysfunction in the fibrotic heart, but may also contribute to the integrity of the cardiac matrix preventing chamber dilation. In addition to the deposition of fibrillar collagens, the extracellular matrix in the remodeling heart exhibits dynamic alterations in its composition that serve to facilitate proliferation and migration of fibroblasts and transduce signals necessary for fibroblast activation. The extent and time course of these alterations are dependent on the underlying etiology of fibrosis.

Because fibrotic cardiac remodeling is associated with both systolic and diastolic dysfunction, prevention and reversal of cardiac fibrosis is an important goal for cardiovascular researchers and clinicians. The present disclosure identifies a therapeutic target for the fibrotic myocardial disease; the effectiveness of the described anti-fibrotic strategy depends on the underlying etiology, the severity and extent of disease, in particular, the treatment at an early/mid CKD stage. In the presence of pro-fibrotic pathophysiologic conditions (such as CKD), protection of the myocardium (cardioprotection) from fibrosis is best achieved by using the composition of the disclosure. For example, an anti-hypertensive treatment or valve surgery would be the ideal strategies for myocardial protection in patients with hypertension or valvular disease, respectively. In contrast, according to the disclosure, the use of the composition is directed to CKD patients, which are not yet suffering from hypertension, but suspected of experiencing fibrotic processes.

Whether established cardiac fibrosis is reversible depends on the etiology and extent of disease, the age of the fibrotic lesions and the amount of protease-resistant cross-linked matrix. In an experimental model of fibrotic interstitial cardiomyopathy due to brief repetitive ischemia/reperfusion, discontinuation of the ischemia protocol resulted in reversal of fibrosis. Both experimental and clinical studies have suggested that hypertensive fibrosis is reversible upon treatment with ACE inhibitors. Lisinopril induced regression of fibrosis in spontaneously hypertensive rats with advanced fibrotic cardiomyopathy. Moreover, in a small clinical study, patients with hypertension, left ventricular hypertrophy and diastolic dysfunction had significant regression of fibrosis (assessed through endomyocardial biopsy) after a 6-month course of lisinopril. Attenuated fibrosis was associated with improved diastolic function.

Reversibility of cardiac fibrosis has also been documented in experimental models of genetic cardiomyopathy. In a mouse model of calcineurin-dependent cardiomyopathy, fibrosis was in part reversed when calcineurin was turned off. In a rabbit model of hypertrophic cardiomyopathy statin therapy induced regression of cardiac fibrosis and hypertrophy. Moreover, AT1 blockade with losartan reversed fibrosis and attenuated TGF-β expression in a transgenic mouse model of human hypertrophic cardiomyopathy. Clearly, established fibrotic lesions due to replacement of a large amount of myocardium are less likely to be reversible.

Although regression of fibrosis has been documented in several cardiac conditions, the mechanisms responsible for reversal of fibrotic disease remain unknown. Clearance of collagen and other matrix proteins from the fibrotic heart likely requires activation of proteases. Whether specific subpopulations of “anti-fibrotic” macrophages and lymphocytes are involved in driving resolution of fibrotic lesions remains unknown. Moreover, the functional characteristics and molecular profile associated with a pro-regression phenotype in cardiac fibroblasts have not been investigated.

EP 14 762 607 describes an implantation of a femoral catheter which increases the presence of heart fibrosis in a 5/6 nephrectomized (5/6-Nx) model to assess the effect of chemical agents on heart fibrosis. Heart fibrosis was determined by the measurement of hydroxyproline (collagen content) and by histological evaluation (HPE and Masson's trichrome staining). However, this animal model represents a late CKD stage, in which hypertension as well as major renal and cardiac damages are elicited. The compositions of the disclosure, in contrast, are directed to preventing myocardial damage and/or application for use in a therapy of cardioprotection at an early/mid CKD stage, in which no hypertension phenotype is observed. In other words, the time point for use of the compositions requires an early stage without structural damage due to hypertension. Only the fibrotic process at an early/mid stage of CKD is subject to the approach of the disclosure.

Fibrosis is an independent predictor of arrhythmogenesis and sudden death in patients. Several established pharmacological therapies that are known to reduce arrhythmias or progression of heart disease affect connective tissue, including ACE inhibitors, aldosterone antagonists and statins. The pharmacological approach of the disclosure is developed to target chronic pathologic processes that lead to the accumulation of fibrotic tissue and heart failure. The use of the composition of the disclosure allows partial or total recovery of fibrosis-induced changes in structural and functional myocardial properties. The present disclosure describes a composition for use in a novel cardioprotective therapy that preserves myocardial muscle, thereby inhibiting its replacement by scar tissue following cardiac injury or insult.

### EXAMPLES

### Example 1—Adenine-Induced Chronic Kidney Disease Animal Model

The experiment was approved by the State Office for Occupational Safety, Consumer Protection and Health (Landesamt für Arbeitsschutz, Verbraucherschutz and Gesundheit Brandenburg) and assigned the animal experiment-number G 2347-31-2015. Male Sprague-Dawley rats (n=66) weighing between 250-280 g were supplied by Envigo (NM Horst, the Netherlands). Rats were housed in cages in groups of 3-4 animals of the same treatment regime and maintained on a 12-hour light/dark cycle. The general condition of each animal was monitored daily. Body weights were measured twice a week. All animals were allowed a few days for acclimatization, for which animals were provided with standard rat chow (C1000 Altromin, Lage, Germany) and water ad libitum. Animals were allocated to the following groups: 1. Healthy vehicle-treated control animals (CNT; n=10); 2. Adenine-induced CKD vehicle-treated animals (CKD; n=14); 3. CKD+low dose (LD) anti-Bone sialoprotein (BSP) antibody (CKD+LD Anti-BSP; n=14); CKD+medium dose (MD) anti-BSP (CKD+MD Anti-BSP; n=14); CKD+high dose (HD) anti-BSP (CKD+HD Anti-BSP; n=14).

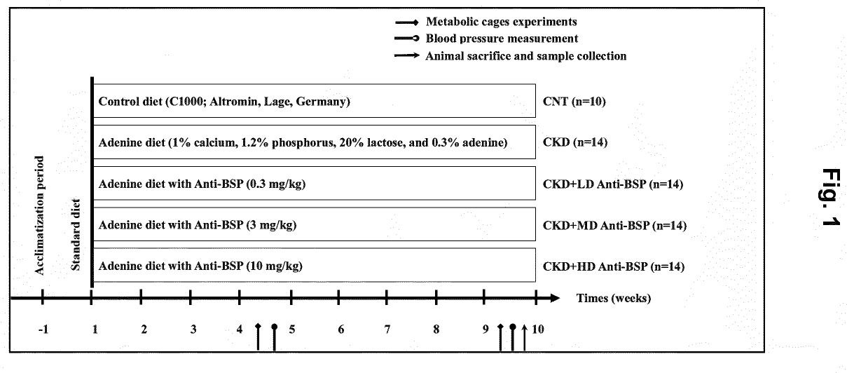

After acclimatization, rats were assigned for ten weeks to a standard chow diet (C1000 Altromin, Lage, Germany), given to the healthy vehicle-treated control animals (CNT; n=10), while for all other groups (CKD; CKD+LD Anti-BSP; CKD+MD Anti-BSP; CKD+HD Anti-BSP; n=56) the chow diet was supplemented with 1% calcium, 1.2% phosphorus, 20% lactose, 19% casein based protein and 0.3% adenine (Altromin Spezialfutter GmbH & Co. KG) in order to induce a model of chronic kidney disease with moderate uremic calcification and without hypertension. Rats were divided into five groups and fed their respective diets for ten weeks. The groups consisted of healthy vehicle-treated control animals (CNT; n=10), adenine-induced CKD vehicle-treated animals (CKD; n=14), and adenine-induced CKD animals treated with anti-BSP-antibody in low (CKD+LD Anti-BSP; n=14), medium (CKD+LD Anti-BSP; n=14), and high concentrations (CKD+HD Anti-BSP; n=14). Animals were started on anti-BSP-antibody treatment concomitantly with their assigned diet; control diet: (C1000 Altromin, Lage, Germany); CKD induction diet (1% calcium, 1.2% phosphorus, 20% lactose, 19% casein-based protein, and 0.3% adenine, Altromin Spezialfutter GmbH & Co. KG); metabolic cage experiment were performed in week 5 and 10; blood pressure measurements were done in week 5 and 10; Animals were sacrificed at the end of the study at week 10; see FIG. 1.

At weeks 5 and 10 of the animal study, the animals were put into metabolic cages for 6 hours to collect urine. The values gathered in these experiments were extrapolated to get 24 hours values. The animals had free accessibility to chow and water. Samples were frozen and stored at −80° C. for later analysis, including measurements of urinary albumin, urinary creatinine, and urinary albumin-creatinine ratio (UACR).

Evaluation of systolic blood pressure—The tail-cuff plethysmography was used to evaluate systolic blood pressure (SBP) at weeks 5 and 10 of the animal study. Each animal was put into a restrainer, a tubular construction from which only the tail of the animal protruded. After that, a blood pressure cuff together with an electronic transducer was fixed to the tail of the animal. The animals were maintained warm using red light set at an appropriate distance. Once the animals were calm and accustomed to the restrainer, blood pressure diagrams were recorded and assessed using Chart™5 (AD Instruments, Sydney, Australia).

Blood and serum collection—At week five, each animal was put into a restrainer, and blood samples were taken by puncturing tail veins without anesthesia. A final blood collection was carried out again at study end at week ten by puncturing the heart under isoflurane anesthesia. After collection, blood samples were incubated at room temperature for between 10-20 minutes and then centrifuged at 4500 g for 10 minutes. Sera were collected, pipetted and stored at −80° C. for later analysis.

Animal sacrifice and organ collection—Animals were sacrificed at week 10. The animals were placed in a box connected to an isoflurane evaporator for the induction of anesthesia, and a mixture of oxygen and isoflurane (3-4%) was introduced into the box. Once the animals were anesthetized, they were taken out of the boxes, and further delivery of the anesthetic was conducted via a head mask. Thoracic and abdominal cavities were opened, and blood samples were withdrawn from the left ventricle, followed by extraction of the heart. Also, the kidneys were harvested, washed gently with normal saline, gently wiped, gently removed the capsule, weighed, and cut longitudinally into two halves. One set of halves was further processed for histological analysis, and the other set of halves was snap-frozen in liquid nitrogen then stored at −80° C. for later analysis. The hearts were also washed gently with normal saline, wiped gently, weighed, and then cut into three parts. Heart apices were flash-frozen in liquid nitrogen; bases were discarded, and the ring-shaped cut-outs below the bases were fixed in 4% formalin. The thoracic aortae were cleaned of adherent tissue, weighed and cut in halves. One set of halves was snap-frozen in liquid nitrogen and the other set was fixed in 4% formalin. Abdominal aortae were frozen in liquid nitrogen-nitrogen. Lungs were harvested, washed gently with normal saline, wiped gently, weighed, and the left wings snap-frozen in liquid nitrogen. Livers were weighed after extraction, and strips of the large lobe of each liver were snap-frozen in liquid nitrogen. The second set of strips was fixed in 4% formalin. The left tibiae were released from muscle tissue and their lengths measured. They were then frozen at −80° C. All muscles attached to the left femur were isolated and excised then frozen at −80° C. for later analysis.

Tissue processing and embedding—Tissue samples were prepared for histological analysis by first fixing them in acid-free (pH 7), 4% phosphate-buffered formaldehyde solutions (Roti®-Histofix 4%, Carl Roth, Karlsruhe, Germany) for twenty-four hours. After fixation in formalin, the samples were dehydrated in several concentrations of ethanol as follows: 24 hours in 70% ethanol, one hour in 96% ethanol and three successive one-hour periods in 100% ethanol. Samples were then cleared in Roticlear® (Carl Roth, Karlsruhe, Germany). The next step was to embed the samples in paraffin (Thermo Scientific Richard-Allen Scientific) for 4 hours in order to produce paraffin blocks. Tissues were first placed in pure molten paraffin type 6 (Thermo Scientific Richard-Allen Scientific) at 56° C. for two hours and then transferred to a second paraffin type 9 (Thermo Scientific Richard-Allen Scientific) bath for an additional two hours. All of these steps were automatically performed overnight in a Shandon Citadel 1000 tissue machine from Thermo Electronics Corporation. The embedding in the histocassettes was then carried out on the Microm EC-350 modular paraffin embedding center from Thermo Scientific surrounding the tissues by paraffin wax, which when cooled and solidified provided sufficient support for section cutting. The paraffin blocks were then cut using a Jung RM 2025 microtome (Leica Biosystems, Wetzlar, Germany) to produce 3-5 μm thick sections on glass slides (Carl Roth, Karlsruhe, Germany). Tissue sections were then placed in a water bath and transferred on glass slides (Carl Roth, Karlsruhe, Germany). Subsequently, the slides were placed in a warming cabinet for 30 minutes to dry and then were stored in a slide box. After they had stored, slides were appropriately stained before conducting a histological examination.

Histological evaluation of renal and cardiac fibrosis using Sirius red-staining—Picrosirius red is a strong anionic dye whose Sirius red component binds to basic groups present within collagen (in the presence of picric acid) resulting in a distinctive red stain (Junqueira et al., 1979). The picric acid component stains the other structures yellow. When applied to kidney and heart tissue, it can, therefore, be used to assess the extent of fibrosis. The slides of kidney and heart tissue sections were stained using this stain. The first step was deparaffinization of the sections by immersing the slides twice in xylol (Carl Roth, Karlsruhe, Germany) for five minutes each time. Then, the sections were rehydrated by immersing the slides in graded ethanol as follows: 100% ethanol for two minutes; 96% ethanol for two minutes; 80% ethanol for two minutes and 70% ethanol for two minutes. The Sirius red staining solution (0.1% w/v) was prepared by dissolving Sirius red (Direct Red 80, Sigma-Aldrich, Missouri, USA) in saturated picric acid solution (1.3% picric acid in the water, Sigma-Aldrich, Missouri, USA). Sections were then stained with Sirius-red by immersing the slides in the Sirius red staining solution for one hour at room temperature and away from direct light. Slides were then washed for a short period of time in 0.01 M HCl and dehydrated by emersion in various concentrations of ethanol as follows: 70% ethanol for two minutes; 80% ethanol for two minutes; 96% ethanol for two minutes; 100% ethanol for two minutes. Finally, the dehydration process was completed by two five-minute emersions in xylol. Sections were then covered with slide covers using Roti®-Mount (Carl Roth, Karlsruhe, Germany).

Collected images were processed and stitched together using BZ-II Analyzer (Keyence, Osaka, Japan) that generated an image across the entire section of renal tissue tissues. Sections were captured using BZ-9000 compact fluorescence microscope (Keyence, Osaka, Japan). The thresholds for detecting the fibrotic area (Sirius red-positive area) and renal tissue area (yellow picric acid positive area) per microscopic field were determined using a random subset of images with the aid of ImageJ software (National Institutes of Health, Bethesda, USA). Then, the percentages of the fibrotic areas per sections were calculated.

Statistical analysis—Statistical analysis in this work was performed using the GraphPad Prism 6 software (La Jolla, Calif., USA). For the statistical analysis of food intake, and body weight, a 2-way (ANOVA) analysis of variance with Bonferroni post-hoc test was performed. Categorical data, such as the presence or absence of calcification in the aorta was tested using Pearson's chi-squared test. Continuous data was checked for normal distribution using the D'Agostino-Pearson normality test. The analysis of variance (ANOVA) test followed by Bonferroni post-hoc test was used for normally distributed data. The Kruskal-Wallis test, followed by Dunn's post-hoc test, was used for non-normally distributed data. In all cases, differences were regarded as statistically significant if P<0.05.

### Example 2—Use of a Monoclonal Anti-BSP Antibody in Adenine-Induced Early/Mid Stage Chronic Kidney Disease Animal Model

Anti-BSP-antibody's stability studies were conducted using HPLC analysis to detect possible aggregation of the anti-BSP-antibody. For the SEC-HPLC (size exclusion chromatography) analysis of possible antibody aggregation, a Shimadzu HPLC device (Kyoto, Japan) was used. It was equipped with an SPD 10AVP (UV-VIS) detector. The eluent was a pH 7.4 sodium phosphate buffer solution (10 mmol/L Na₂HPO₄, 1.8 mmol/L Na₂HPO₄, 0.9% NaCl, 0.02% NaN₃, all obtained from Carl Roth, Karlsruhe, Germany). Proteins were separated via a Tosohaas tsk gel column (TosoHaas GmbH, G4000 PWXL, 6 μm, ID 7.5 mm, Length 20 cm, Griesheim, Germany) at a flow rate of 0.5 mL/min and an injection volume of 20 μL. The column temperature was held at 30° C. Protein absorbance was detected at both 254 and 280 nm. The total analysis time, conducted in an isocratic mode, was 60 minutes per run.

To assess the prophylactic effects of the anti-BSP-antibody on myocardial fibrosis, anti-BSP-antibody treatment was started in animals concomitantly with their assigned diets. The anti-BSP-antibody was supplied from the manufacturer Immundiagnostik AG (Bensheim, Germany; see EP15756875.9) with phosphate-buffered saline (PBS) as a vehicle in aliquots of the same lot to avoid freeze-thaw cycles. Animals either received twice-weekly subcutaneous injections (sc) of the anti-BSP-antibody in low (0.3 mg/kg), medium (3 mg/kg), and high (10 mg/kg) doses, or were treated with comparable volumes of phosphate-buffered saline (PBS). Anti-BSP-antibodies were kept frozen immediately after arrival at −20° C. At the beginning of the animal study, the antibody solution was thawed on ice. Before injection into the animals, it was warmed to rat body temperature. Animals were subcutaneously injected with their assigned doses of anti-BSP-antibody twice weekly (every Monday and Thursday) for a period of ten weeks. Depending on body weight, between 0.7 ml and 1.1 ml of anti-BSP-antibody were administered per animal.

### Example 3—Effects of Use of Anti-BSP-Antibody Treatment on Food Intake and Body Weight Changes Throughout the Study

Food intake and body weights of the animals were recorded repetitively in a period of ten weeks according to the study protocol shown in FIG. 1.

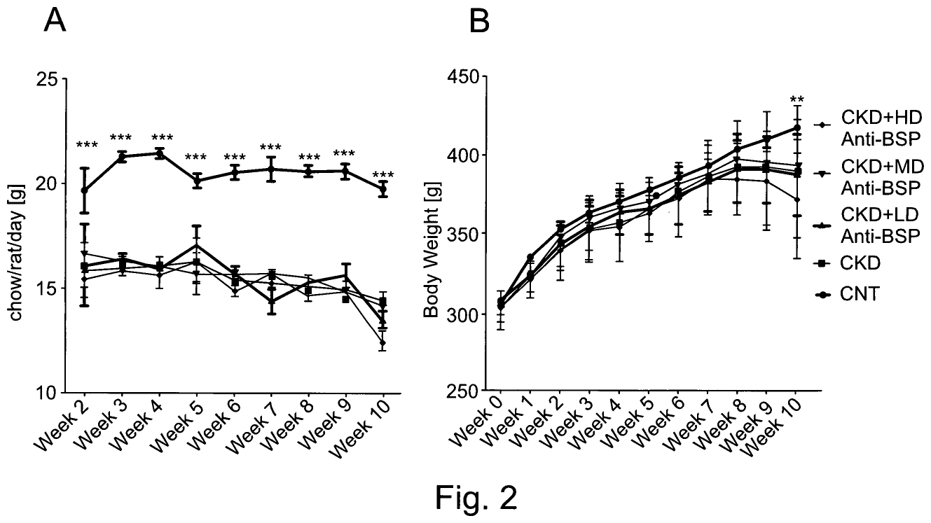

FIG. 2 shows a schematic representation of the values of food intake and body weight changes throughout the study. FIG. 2A: Food intake; the food intake of diseased adenine-induced CKD vehicle-treated animals (CKD; n=14) was significantly reduced compared to healthy vehicle-treated control animals (CNT; n=10) animals; Treatment with the Anti-BSP-antibody in low (CKD+LD Anti-BSP; n=14), medium (CKD+LD Anti-BSP; n=14), and high concentrations (CKD+HD Anti-BSP; n=14) did not significantly impact on food intake compared with adenine-induced CKD vehicle-treated animals. FIG. 2B: Body weight throughout the study; average body weights decreased over time and at week ten were significantly decreased compared with that of healthy vehicle-treated control animals (CNT; n=10) animals. Treatment with the Anti-BSP-antibody did not significantly affect body weight. Differences between the groups were assessed by two-way analysis of variance followed by a Bonferroni post-hoc test. Values are given as mean±SEM; **P<0.01; ***P<0.001 versus CKD.

No significant difference in body weight was found among the groups until week nine. However, by week 10 (at the end of the study), the healthy control animals had reached significantly higher weights than all CKD groups (FIG. 2B). Treatment with the anti-BSP-antibody did not significantly affect the food intake or the body weights of the animals compared to CKD vehicle-treated animals.

### Example 4—Effects of Use of Anti-BSP-Antibody on Absolute and Relative Organ Weights

Table 1 shows absolute and relative organ weights of healthy vehicle-treated control animals (CNT; n=10), adenine-induced CKD vehicle-treated animals (CKD; n=14), and adenine-induced CKD animals treated with Anti-BSP-antibody in low (CKD+LD Anti-BSP; n=14), medium (CKD+LD Anti-BSP; n=14), and high concentrations (CKD+HD Anti-BSP; n=14), which were calculated by dividing the absolute organ weight by either body weight [g] or tibial length [mm]. In normally distributed data, differences between the groups were assessed by one-way analysis of variance followed by a Bonferroni post-hoc test. For not normally distributed data, the Kruskal-Wallis test followed by a Dunn's post-hoc test was used. Values are given as mean±SEM; **P<0.01; ***P<0.001 versus CTN. P<0.05; P<0.01, versus CKD. BW: body weight; TL: tibia length

Compared with the healthy vehicle-treated control animals, adenine-induced CKD vehicle-treated animals showed a significant increase in the average weight of the right kidney and the average weight of the left kidney even after adjusting the absolute weights to tibial length and body weight. Compared with the adenine-induced CKD vehicle-treated animals, animals treated with medium dose of the anti-BSP-antibody displayed a significant decrease in the average absolute and relative, body weight and tibial length adjusted weights of the right kidney. Compared with the adenine-induced CKD vehicle-treated animals, animals treated with high dose of the anti-BSP-antibody displayed a significant decrease in the average relative weights of the right kidney after correction using body weight. Treatment with the anti-BSP-antibody did not significantly affect the average weight of the left kidney. The livers of adenine-induced CKD vehicle-treated animals were found to weigh significantly less than the livers of the healthy vehicle-treated control animals (Table 1). Treatment with the anti-BSP-antibody did not significantly affect liver weight. These findings were affirmed after liver weight correction using body weight and tibial length. The heart, lung, and aorta displayed no differences regarding their absolute and relative weights among the study groups.

TABLE 1
Absolute and relative organ weights of the study groups

| | | | CKD + LD | CKD + MD | CKD + HD |
|---|---|---|---|---|---|
| | CNT | CKD | Anti-BSP | Anti-BSP | Anti-BSP |
| Heart [g] | 1.14 ± 0.03 | 1.10 ± 0.02 | 1.14 ± 0.04 | 1.07 ± 0.03 | 1.05 ± 0.03 |
| Liver [g] | 12.26 ± 0.25 | 10.44 ± 0.41 ** | 11.05 ± 0.31 | 10.30 ± 0.33 | 9.73 ± 0.41 |
| Lung [g] | 0.54 ± 0.01 | 0.53 ± 0.01 | 0.55 ± 0.02 | 0.49 ± 0.04 | 0.51 ± 0.02 |
| Left kidney [g] | 1.16 ± 0.02 | 2.69 ± 0.14 *** | 2.65 ± 0.15 | 2.58 ± 0.15 | 2.76 ± 0.09 |
| Right kidney [g] | 1.17 ± 0.02 | 1.82 ± 0.07 *** | 1.73 ± 0.04 | 1.57 ± 0.05 ^{##} | 1.61 ± 0.06 ^{#} |
| Aorta [g] | 0.07 ± 0.007 | 0.09 ± 0.005 | 0.09 ± 0.006 | 0.08 ± 0.004 | 0.1 ± 0.015 |
| Body weight [g] | 418.6 ± 5.60 | 390.4 ± 11.33 | 392.7 ± 6.91 | 394.5 ± 7.92 | 368.9 ± 9.79 |
| Heart/BW [g/g] *10000 | 27.31 ± 0.81 | 28.37 ± 0.71 | 29.14 ± 1.02 | 27.14 ± 0.65 | 28.52 ± 0.54 |
| Liver/BW [g/g] *10000 | 292.7 ± 3.94 | 266.8 ± 5.60 ** | 281.1 ± 5.16 | 260.7 ± 5.08 | 262.9 ± 6.08 |
| Lung/BW [g/g] *10000 | 12.83 ± 0.14 | 13.66 ± 0.33 | 14.04 ± 0.43 | 12.34 ± 0.87 | 13.88 ± 0.26 |
| Left kidney/BW [g/g] *10000 | 27.77 ± 0.43 | 69.37 ± 4.44 | 67.62 ± 4.05 | 65.72 ± 3.91 | 75.6 ± 3.42 |
| Right kidney/BW [g/g] *10000 | 28.08 ± 0.55 | 47.36 ± 2.58 *** | 44.19 ± 1.09 | 39.79 ± 1.17 ^{##} | 43.93 ± 1.89 |
| Aorta/BW [g/g] *10000 | 1.77 ± 0.17 | 2.34 ± 0.17 | 2.34 ± 0.15 | 2.16± 0.13 | 2.64 ± 0.46 |
| Tibia length [mm] | 41.72 ± 0.26 | 41.27 ± 0.37 | 41.84 ± 0.22 | 41.55 ± 0.30 | 41.45 ± 0.28 |
| Heart/TL [g/mm] * 1000 | 27.37 ± 0.74 | 26.65 ± 0.50 | 27.34 ± 1.01 | 25.76 ± 0.77 | 25.31 ± 0.64 |
| Liver/TL [g/mm] | 0.29 ± 0.01 | 0.25 ± 0.01 ** | 0.26 ± 0.01 | 0.25 ± 0.01 | 0.23 ± 0.01 |
| Lung/TL [g/mm] * 1000 | 12 .87 ± 0.21 | 12.86 ± 0.33 | 13.15 ± 0.40 | 11.74 ± 0.85 | 12.32 ± 0.32 |
| Left kidney/TL [g/mm] *1000 | 27.85 ± 0.44 | 65.2.7 ± 3.52 *** | 63.26 ± 3.71 | 61.98 ± 3.37 | 66.56 ± 2.22 |
| Right kidney/TL [g/mm] *1000 | 28.14 ± 0.43 | 44.24 ± 1.86 *** | 41.38 ± 0.93 | 37.68 ± 1.07 ^{##} | 38.83 ± 1.50 * |
| Aorta/TL [g/mm] *1000 | 1.78 ± 0.17 | 2.17 ± 0.12 | 2.19 ± 0.14 | 2.04 ± 0.11 | 2.29 ± 0.36 |

### Example 5—Effects of Use of Anti-BSP-Antibody on Cardiac Fibrosis and Systolic Blood Pressure

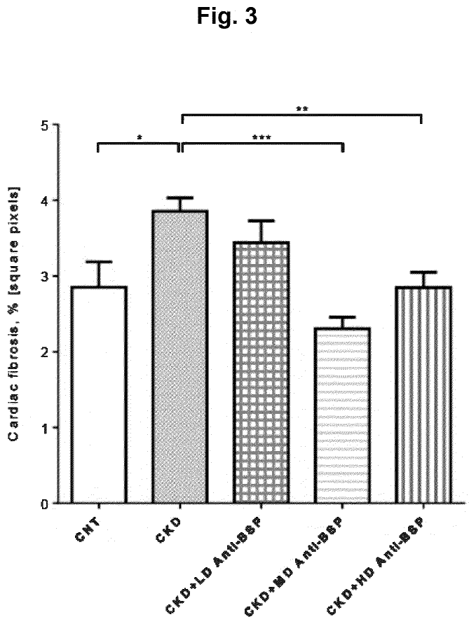

FIG. 3 describes the effect of the use of anti-BSP-antibody on cardiac fibrosis of different study groups. Quantification of cardiac interstitial collagen content was investigated by analyzing microscopic images of Sirius red stained heart sections. Results are expressed as the ratio of collagen area to heart area. Adenine-induced CKD vehicle-treated animals (CKD; n=14) displayed a significantly higher fibrosis compared with healthy vehicle-treated control animals (CNT; n=10). Animals treated with the Anti-BSP-antibody in medium (CKD+LD Anti-BSP; n=14) and high concentrations (CKD+HD Anti-BSP; n=14) had a significantly lower fibrosis compared with adenine-induced CKD vehicle-treated animals. Treatment with low doses of the Anti-BSP-antibody (CKD+LD Anti-BSP; n=14) did not significantly affect the fibrosis percent. Differences between the groups were assessed by one-way analysis of variance followed by a Bonferroni post-hoc test. Values are given as mean±SEM; *P<0.05; **P<0.01; ***P<0.001 versus CKD.

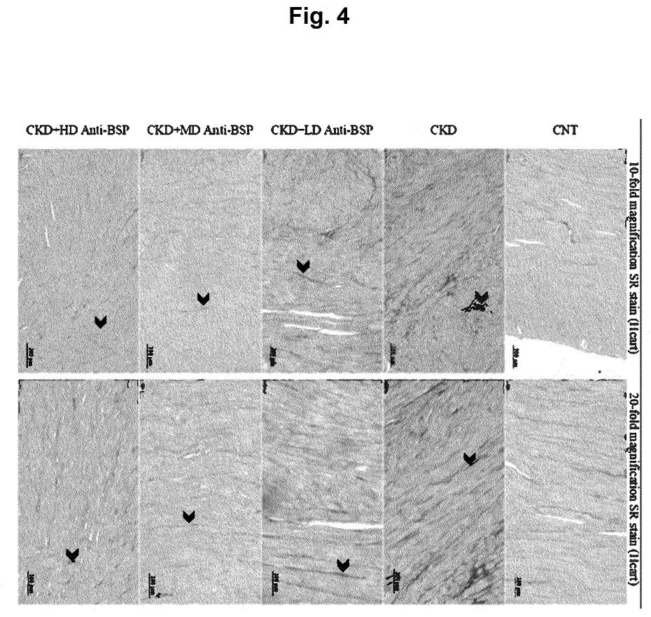

FIG. 4 shows representative images of Sirius red-stained myocardium sections. Representative images of Sirius red-stained myocardium sections of healthy vehicle-treated control animals (CNT), adenine-induced CKD vehicle-treated animals (CKD), and adenine-induced CKD animals treated with Anti-BSP-antibody in low (CKD+LD Anti-BSP), medium (CKD+MD Anti-BSP), and high concentrations (CKD+HD Anti-BSP). Red (arrow, dark area) indicates collagen fiber, and yellow (light area) indicates myocardium. Adenine-induced CKD vehicle-treated animals associated with a remarkable accumulation of Sirius red-positive collagen (chevron) as compared to healthy vehicle-treated control rats.

Systolic blood pressure was assessed (SBP) via tail-cuff measurement at week 5 and week 10 of the experiment. Measurements yielded no significant differences between the groups, as shown in Table 2.

TABLE 2
Systolic blood pressure

| | | | CKD + LD | CKD + MD | CKD + HD |
|---|---|---|---|---|---|
| | CNT | CKD | Anti-BSP | Anti-BSP | Anti-BSP |
| SBP Week 5 [mmHg] | 140.9 ± 4.07 | 140.4 ± 3.71 | 140.4 ± 3.71 | 145.3 ± 2.93 | 145.4 ± 3.53 |
| SBP Week 10 [mmHg] | 140.8 ± 4.08 | 145.5 ± 3.60 | 140.4 ± 2.30 | 145.7 ± 3.91 | 146.1 ± 4.26 |

### Example 6—Effects of Use of Anti-BSP-Antibody on Liver Function and Liver Histology

To assess liver function, serum albumin and C-reactive protein (CRP) were performed to assess the malnutrition and the inflammatory condition associated with CKD. Levels of aspartate aminotransferase (AST/GOT) and alanine aminotransferase (ALT/GPT) in serum measured in addition to HE staining of liver sections to assess the condition of the liver. Serum albumin, CRP, GOT, and GPT were performed in collaboration with Immundiagnostik AG (Bensheim, Germany).

Histological examination of H&E-stained liver sections revealed mild inflammatory infiltration in the portal areas in Adenine-induced CKD animals (not shown). Adenine-induced CKD vehicle-treated animals had significantly lower serum albumin levels than the healthy vehicle-treated control animals. Serum concentrations of the liver produced inflammation marker C-reactive protein (CRP) was significantly higher in adenine-induced CKD vehicle-treated animals than in healthy vehicle-treated control animals, indicating a systemic inflammatory process. However, no significant differences between the adenine-induced CKD animals and the anti-BSP-antibody-treated animals were observed. Serum levels of glutamic oxaloacetic transaminase (GOT), glutamic pyruvic transaminase (GPT) were measured, but no significant differences were observed among the various study groups.

### Example 7—Effects of Use of Anti-BSP-Antibody on Renal Function Parameters in Adenine-Induced Early/Mid Stage Chronic Kidney Disease Animal Model

Measurements of 24-hour urinary volume, urinary pH, serum cystatin C levels, serum and urinary creatinine, 24-hour urinary albumin excretion, GFR, and urinary albumin-to-creatinine ratio (UACR) were performed to assess the level of renal insufficiency and document any measurable uremia. Urinary albumin, serum and urinary creatinine conducted in collaboration with Immundiagnostik AG (Bensheim, Germany). Levels of urinary creatinine were determined quantitatively using a creatinine detection kit from Immundiagnostik AG (Bensheim, Germany).

Urinary albumin was determined using an albumin detection ELISA kit from Immundiagnostik AG (Bensheim, Germany). Levels of serum cystatin C, a marker of the rate of glomerular filtration, were measured using a solid-phase sandwich ELISA (Quantikine® Mouse/Rat Cystatin C ELISA kit, R&D Systems, MSCTC0). For each rat, the twenty-four-hour albumin excretion was calculated by multiplying the urinary albumin level of each sample by the volume of urine excreted by the same rat in 24 hours. UACR was calculated by dividing urinary albumin levels by urinary creatinine levels. GFR was calculated from serum and urinary creatinine according to the formula “urinary creatinine*urine volume/serum creatinine.” GFR was then corrected for body weights.

Measurements of 24-hour urinary volume, urinary pH, serum cystatin C levels, serum and urinary creatinine, 24-hour urinary albumin excretion, GFR, and urinary albumin-to-creatinine ratio (UACR) were performed to assess the level of renal insufficiency and document any measurable uremia. The adenine-induced CKD vehicle-treated animals had a significantly higher average 24-hour urine output and significantly lower urinary pH levels compared with the healthy vehicle-treated control animals. Urinary albumin excretion was measured in urine collected at week 10 of the animal experiment. A non-significant increase in the average 24-hour urinary albumin excretion of adenine-induced CKD vehicle-treated animals compared with the healthy vehicle-treated control animals was observed. Compared with the healthy vehicle-treated control animals, the adenine-induced CKD vehicle-treated animals excreted significantly lower levels of urinary creatinine. Calculated differences in UACR were all non-significant among the groups. Serum cystatin C is commonly used as a measure of renal function. In this study, serum cystatin C levels in the adenine-induced CKD vehicle-treated animals were significantly higher than that in control vehicle-treated animals. The adenine-induced CKD vehicle-treated animals showed significantly higher levels of serum creatinine compared with the healthy vehicle-treated control animals. GFR was calculated from serum and urinary creatinine according to the formula “urinary creatinine*urine volume/serum creatinine.” GFR was then corrected for body weights. Average GFR was significantly lower in the adenine-induced CKD vehicle-treated animals compared with the control vehicle-treated animals. There were, however, no significant differences in any of the above-mentioned parameters between the adenine-induced CKD vehicle-treated animals and the adenine-induced early/mid stage CKD anti-BSP-antibody-treated animals. Study group specific results of the analyzed renal function parameters are shown in Table 3.

Table 3 shows the results of different renal parameters of healthy vehicle-treated control animals (CNT; n=10), adenine-induced CKD vehicle-treated animals (CKD; n=14), and adenine-induced CKD animals treated with Anti-BSP-antibody in low (CKD+LD Anti-BSP; n=14), medium (CKD+LD Anti-BSP; n=14), and high concentrations (CKD+HD Anti-BSP; n=14). In normally distributed data, differences between the groups were assessed by one-way analysis of variance followed by a Bonferroni post-hoc test. For not normal distributed data, the Kruskal-Wallis test followed by a Dunn's post-hoc test was used. Values are given as mean±SEM; ***P<0.001 versus CKD; x measured in collaboration with Immundiagnostik AG, Bensheim, Germany.

TABLE 3
Assessment of renal function parameters in the study groups

| | | | CKD + LD | CKD + MD | CKD + HD |
|---|---|---|---|---|---|
| | CNT | CKD | Anti-BSP | Anti-BSP | Anti-BSP |
| Urine mL/24 h (week 10) | 6.50 ± 0.83 | 45.61 ± 2.91 *** | 44.46 ± 3.50 | 43.95 ± 2.64 | 45.66 ± 3.07 |
| Urine pH ^{x} | 8.08 ± 0.16 | 5.41 ± 0.03 *** | 5.46 ± 0.03 | 5.47 ± 0.03 | 5.49 ± 0.08 |
| Urine albumin [μg/24 h] ^{x} | 147.2 ± 40.80 | 399.6 ± 106.8 | 608.8 ± 148.7 | 698.9 ± 163.9 | 668.8 ± 208.5 |
| Urine creatinine [mg/dL] ^{x} | 177.2 ± 24.47 | 25.79 ± 1.59 *** | 25.79 ± 1.48 | 26.21 ± 2.03 | 24.71 ± 1.71 |
| Urine UACR [μg/mg] | 1.36 ± 0.33 | 3.23 ± 0.71 | 5.00 ± 1.05 | 7.01 ± 2.22 | 5.94 ± 1.19 |
| Serum Cystatin C [ng/mL] | 2256 ± 141.4 | 7908 ± 746.2 *** | 7218 ± 595.1 | 6596 ± 575.0 | 9100 ± 1007 |
| Serum creatinine [mg/dL] ^{x} | 0.34 ± 0.01 | 1.60 ± 0.14 *** | 1.47 ± 0.14 | 1.45 ± 0.15 | 2.10 ± 0.22 |
| GFR [mL/24 h]/body weight [g] | 8.34 ± 1.38 | 2.15 ± 0.29 *** | 2.13 ± 0.28 | 2.39 ± 0.29 | 1.83 ± 0.269 |

### Example 8—Bone Sialoprotein Expression in Thoracic Aortic Tissue

Evaluation of aortic BSP expression and representative photomicrographs of BSP immunofluorescence-stained aortae was performed (not shown). Adenine-induced CKD vehicle-treated animals (CKD; n=14) displayed a significantly higher aortic BSP expression compared with healthy vehicle-treated control animals (CNT; n=10). Treatment with the Anti-BSP-antibody in low (CKD+LD Anti-BSP; n=14), medium (CKD+MD Anti-BSP; n=14) and high concentrations (CKD+HD Anti-BSP; n=14) concentrations did not significantly affect aortic BSP expression. Differences between the groups were assessed by the Kruskal-Wallis test, followed by Dunn's post-hoc test. Values are given as mean±SEM; **P<0.01 versus CKD.

1-10. (canceled)

11. A method of treating a subject diagnosed or suspected of suffering from cardiac fibrosis, comprising the administration of a medicinal preparation containing a monoclonal antibody against bone sialoprotein (BSP), whereby a pathological accumulation of collagen and/or the progression of fibrosis are prevented.

12. The method of claim 11, wherein the subject is human and suffers from early/mid-stage chronic kidney disease.

13. The method of claim 11, wherein the subject is suspected of suffering from cardiac fibrosis.

14. The method of claim 11, wherein the medicinal preparation is administered to prevent fibrosis and/or accumulation of collagen in the myocardium.

15. The method of claim 11, wherein the subject suffers from uremic calcification.

16. The method of claim 11, wherein the subject does not suffer from hypertension.

17. The method of claim 11, wherein the medicinal preparation is administered in a therapeutically effective amount between 0.1 to 10 mg/kg body weight, preferably 1 to 5 mg/kg body weight, more preferably 2.5 to 3.5 mg/kg body weight.

18. The method of claim 11, wherein the medicinal preparation comprises a pharmaceutically acceptable vehicle and is administered subcutaneously.

19. The method of claim 11, wherein the antibody is a rat monoclonal antibody or a humanized monoclonal antibody.

20. A medicinal preparation that comprises a monoclonal antibody against bone sialoprotein (BSP) for cardioprotection therapy in a subject diagnosed or suspected of suffering from fibrosis.
